(19) United States
(12) Patent Application Publication
(10) Pub. No.: US 2017/0362586 A1
(43) Pub. Date: Dec. 21, 2017

(54) METHODS AND KITS FOR PURIFYING PLASMID DNA

(71) Applicant: Corning Incorporated, Corning, NY (US)

(72) Inventors: Yi-Cheng Hsieh, Horseheads, NY (US); Cheng-l Hsu, Zhubei city (TW); Jun Min Hu, Suzhou City (CN); Chia-Ling Wu, Tainan City (TW); Hsan Jan Yen, New Taipei City (TW)

(21) Appl. No.: 15/533,547

(22) PCT Filed: Dec. 9, 2015
(86) PCT No.: PCT/US15/64598

§ 371 Date: Jun. 6, 2017

Related U.S. Application Data
(60) Provisional application No. 62/089,333, filed on Dec. 9, 2014.

(51) Int. Cl. C12N 15/10

(52) U.S. Cl. CPC C12N 15/1013

(57) ABSTRACT

The disclosure relates to methods for nucleic acid purification, comprising (a) combining a sample comprising at least one nucleic acid with a suspension buffer to form a suspension, (b) combining the suspension with a lysis buffer to form a lysate, (c) combining the lysate with a binding buffer to form a solution; (d) combining the solution with at least one magnetic particle to form a combined solution comprising at least one modified magnetic particle reversibly bound to the at least one nucleic acid, (e) separating the at least one modified magnetic particle from the combined solution, (f) washing the at least one modified magnetic particle with a first wash buffer and a second wash buffer, and (g) combining the at least one modified magnetic particle with an elution buffer. Kits comprising these buffers and magnetic particles are also disclosed herein.

This application claims the benefit of priority under 35 U.S.C. §119 of U.S. Provisional Application Ser. No. 62/089333 filed on Dec. 9, 2014 the content of which is relied upon and incorporated herein by reference in its entirety.

### FIELD OF THE DISCLOSURE

The present disclosure relates generally to methods and kits for nucleic acid purification and, more particularly, to magnetic particle-based kits and methods for purifying transfection-grade plasmid DNA.

### BACKGROUND

Nucleic acid purification, such as the isolation of DNA or RNA, can be an important step in various biochemical and diagnostic procedures. Plasmid DNA (pDNA) can be used in many applications from preparing vectors for cloning to generating templates for transcription or coupled transcription/translation reactions. It may be necessary to separate nucleic acids from the complex mixtures in which they are often found (e.g., tissues, cells, fluids, etc.) before additional processing, such as cloning, transfection, sequencing, amplification, hybridization, and/or synthesis, etc. can be performed. The presence of contaminating materials, e.g., proteins, lipids, and/or carbohydrates, can impede or prevent many of these procedures. For example, impurities carried over during isolation can lower yields and/or prevent enzyme systems from synthesizing the product of interest. In addition, DNA may also contaminate RNA applications, and vice versa. Thus, it can be important to effectively isolate nucleic acids from various diverse starting materials to ensure a desired end-use functionality.

The selected method for purifying nucleic acids can, in some instances, impact various properties of the isolated product, including yield, quality, and/or purity of the nucleic acid sample. While many approaches have been developed for nucleic acid purification, these methods may have one or more drawbacks including, for example, high cost, high complexity, slow speed, low yield, low purity, contamination, toxicity, and/or inefficiency. Magnetic particles have previously been used to bind and separate nucleic acids in various purification methods. These methods may have several advantages, such as eliminating centrifugation and/or vacuum processing steps, yielding a higher purity product, and or improving operator safety. However, methods employing magnetic particles can still suffer from various disadvantages, such as slow speed, high complexity, and/or poor overall yield.

Accordingly, it would be advantageous to provide magnetic particle-based methods and kits for nucleic acid purification that may be faster, less complex, less expensive, and/or improved in terms of product purity and/or yield. The resulting purified nucleic acids can be used in a wide variety of biochemical and diagnostic applications, such as polymerase chain reactions (PCR), cloning, transfection, restriction digest, and clinical diagnostics.

### SUMMARY

The disclosure relates, in various embodiments, to methods for purifying nucleic acids, the methods comprising (a) combining a sample comprising at least one nucleic acid with a suspension buffer to form a suspension, (b) combining the suspension with a lysis buffer to form a lysate, (c) combining the lysate with a binding buffer to form a solution; (d) combining the solution with at least one magnetic particle to form a combined solution comprising at least one modified magnetic particle reversibly bound to the at least one nucleic acid, (e) separating the at least one modified magnetic particle from the combined solution, (f) washing the at least one modified magnetic particle with a first wash buffer and a second wash buffer, and (g) combining the modified magnetic particle with an elution buffer.

According to various embodiments, the suspension buffer can comprise at least one ion chelating agent, present in a concentration ranging from about 1 mM to about 10 mM, and at least one buffer compound, present in a concentration ranging from about 10 mM to about 100 mM. The lysis buffer may comprise at least one detergent chaotropic agent, present in a concentration ranging from about 1% to about 10% by weight, and at least one buffer compound, present in a concentration greater than or equal to about 0.2 M. In non-limiting embodiments, the binding buffer can comprise at least one chaotropic agent, present in a concentration ranging from about 4 M to about 6 M, optionally at least one salt, present in a concentration ranging from about 0.1 M to about 2 M, at least one alcohol, present in a concentration ranging from about 1% to about 5% by volume, and at least one buffer compound, present in a concentration ranging from about 0.25% to about 3% by weight. According to additional embodiments, the first wash buffer can comprise at least one chaotropic agent, present in a concentration ranging from about 4 M to about 6 M, at least one ion chelating agent, present in a concentration ranging from about 1 mM to about 10 mM, at least one buffer compound present in a concentration ranging from about 10 mM to about 100 mM, and at least one alcohol, present in a concentration ranging from about 30% to about 50% by volume. In further embodiments, the second wash buffer may comprise at least one alcohol and optionally at least one salt. Magnetic particles can be chosen, for example, from carboxyl coated magnetic particles, silica-based magnetic particles, and combinations thereof. Also disclosed herein are nucleic acid purification kits comprising these buffers and magnetic particles.

Additional features and advantages of the invention will be set forth in the detailed description which follows, and in part will be readily apparent to those skilled in the art from that description or recognized by practicing the invention as described herein, including the detailed description which follows, the claims, and the appended drawings.

It is to be understood that both the foregoing general description and the following detailed description present various embodiments of the disclosure, and are intended to provide an overview or framework for understanding the nature and character of the claims. The accompanying drawings are included to provide a further understanding of the disclosure, and are incorporated into and constitute a part of this specification. The drawings illustrate various embodiments of the disclosure and together with the description serve to explain the principles and operations of the invention.

### BRIEF DESCRIPTION OF THE DRAWINGS

The following detailed description can be best understood when read in conjunction with the following drawings, where like structures are indicated with like reference numerals and in which:

FIG. 1 is a flow diagram illustrating a nucleic acid purification method according to one embodiment of the disclosure;

FIG. 2 is a graph illustrating pDNA yield for methods according to various embodiments of the disclosure as compared to a prior art method;

FIG. 3 is a graph illustrating transfection efficiency for methods according to various embodiments of the disclosure as compared to a prior art method;

FIG. 4 illustrates an agarose gel electrophoresis analysis of pDNA (cut or uncut with EcoRI) purified using methods according to various embodiments of the disclosure and using a prior art method;

FIG. 5 is a graph illustrating pDNA yield for methods according to various embodiments of the disclosure as compared to a prior art method; and

FIG. 6 is a graph illustrating transfection efficiency for methods according to various embodiments of the disclosure as compared to a prior art method.

### DETAILED DESCRIPTION

### Methods

Disclosed herein are methods for nucleic acid purification, the methods comprising (a) combining a sample comprising at least one nucleic acid with a suspension buffer to form a suspension, (b) combining the suspension with a lysis buffer to form a lysate, (c) combining the lysate with a binding buffer to form a solution; (d) combining the solution with at least one magnetic particle to form a combined solution comprising at least one modified magnetic particle reversibly bound to the at least one nucleic acid, (e) separating the at least one modified magnetic particle from the combined solution, (f) washing the at least one modified magnetic particle with a first wash buffer and a second wash buffer, and (g) combining the at least one modified magnetic particle with an elution buffer.

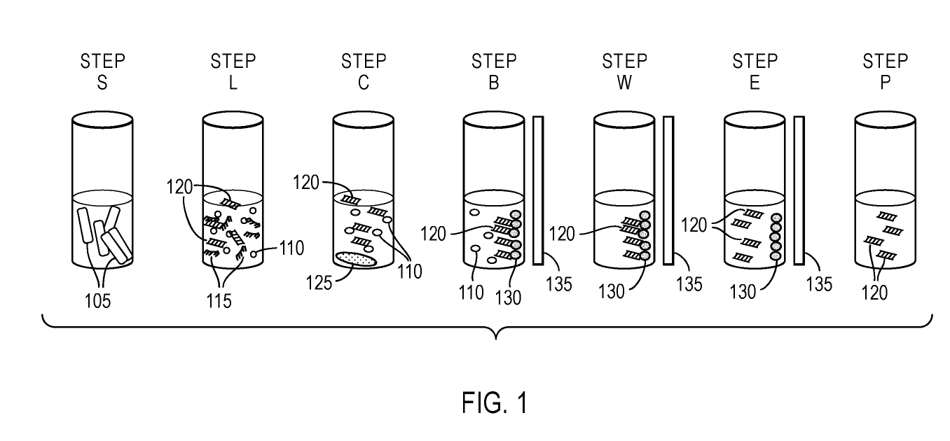

Embodiments of the disclosure will be discussed with reference to FIG. 1, which illustrates a flow diagram for a nucleic acid purification method according to non-limiting embodiments of the disclosure. The following general description is intended to provide an overview of the claimed methods and various aspects will be more specifically discussed throughout the disclosure with reference to the non-limiting embodiments, these embodiments being interchangeable with one another in the context of the general method discussed below.

As demonstrated in FIG. 1, a sample (e.g., bacteria) 105 comprising at least one nucleic acid can be suspended in a suspension buffer in step S. The sample may then be lysed in step L to produce a lysate comprising pDNA 120 and various contaminants 110, including undesired nucleic acids 115 (such as RNA and genomic DNA). The lysate can further be combined and incubated with a binding/neutralization buffer, and processed in step C by centrifugation to separate out any agglomerates or macromolecules 125. Magnetic particles 130 can then be added to the remaining solution. pDNA 120 released from the sample (bacteria) 105 may be reversibly bound to the surface of the magnetic particles 130 in step B. A magnet 135 can be used to attract the magnetic particles 130 and separate them from the remaining solution. Unbound contaminants 110 can be removed by washing the magnetic particles 130 using one or more washing buffers in step W. The pDNA 120 can then be eluted and unbound from the magnetic particles using an elution buffer in step E. The magnetic particles 130 can be removed from the solution in step P, e.g., using a magnet, to produce a purified pDNA product which can then be used in a variety of applications.

As used herein, the term “sample comprising at least one nucleic acid” and variations thereof is intended to denote any material, such as a specimen or culture, obtained from biological or environmental samples, which may contain at least one nucleic acid, e.g., a DNA molecule, RNA molecule, or DNA/RNA hybrid molecule. The at least one nucleic acid can include, for example, genomic DNA, chromosomal DNA (cDNA), plasmid DNA (pDNA), total RNA, messenger RNA (mRNA), ribosomal RNA (rRNA), transfer RNA (tRNA), and/or RNA/DNA hybrids. Biological samples can include human and animal samples, such as cells, tissues, and bodily fluids, e.g. urine, whole blood, and blood-derived fluids such as serum and plasma. Environmental samples can include plant tissues, such as agarose, and bacterial samples, to name a few. In various embodiments, the sample can comprise a bacterial culture. For example, the bacterial culture can be prepared overnight at a concentration of O.D. 600 approximately equal to 1 (8×10⁸ cells/ml). According to additional embodiments, the at least one nucleic acid to be purified can be DNA, e.g., pDNA.

The sample comprising the at least one nucleic acid can be combined, e.g., suspended, in a suspension buffer (S1) (see step S in FIG. 1). The suspension buffer S1 can comprise, in various embodiments, at least one ion chelating agent (IC1) and at least one buffer compound (B1). The suspension buffer S1 can also further include RNase, in a concentration ranging from about 10 μg/ml to about 1,000 μg/ml, such as from about 50 μg/ml to about 500 μg/ml, or from about 100 μg/ml to about 250 μg/ml, including all ranges and subranges in between. In certain embodiments, RNase can be added to the suspension buffer S1 prior to combination with the sample.

The at least one ion chelating agent IC1 can be present in the suspension buffer S1 in a concentration ranging, for example, from about 1 mM to about 10 mM, such as from about 2 mM to about 9 mM, from about 3 mM to about 8 mM, from about 4 mM to about 7 mM, or from about 5 mM to about 6 mM, including all ranges and subranges therebetween. For example, the IC1 concentration can be about 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, or 10 mM, including all ranges and subranges therebetween. According to various embodiments, the at least one ion chelating agent IC1 can be chosen from ethylenediamine tetraacetic acid (EDTA) and isomers thereof such as ethylenediamine-N,N′-disuccinic acid (EDDS); cyclohexane-1,2-diaminetetraacetic acid (CDTA); iminodisuccinic acid (IDS); polyaspartic acid (PASA); methylglycinediacetic acid (MGDA); L-glutamic acid N,N-diacetic acid, tetrasodium salt (GLDA), and combinations thereof. In certain non-limiting embodiments, the at least one ion chelating agent IC1 can be chosen from EDTA and isomers thereof. The at least one ion chelating agent IC1 may, in various embodiments, also serve as a protease inhibitor.

The at least one buffer compound B1 can be present in the suspension buffer S1 in a concentration ranging, for instance, from about 10 mM to about 100 mM, such as from about 20 mM to about 90 mM, from about 30 mM to about 80 mM, from about 40 mM to about 70 mM, or from about 50 mM to about 60 mM, including all ranges and subranges therebetween. For example, the B1 concentration can be about 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM, including all ranges and subranges therebetween. According to various embodiments, the at least one buffer compound B1 can be chosen from Tris, Tris-HCl, 3-(N-morpholino)propanesulfonic acid (MOPS), 4-(2-hydroxyethyl)-1-piperazineethanesulfonic acid (HEPES), 2-(N-morpholino)ethanesulfonic acid (MES), and combinations thereof. In certain non-limiting embodiments, the at least one buffer compound B1 can be chosen from Tris and Tris-HCl.

The at least one buffer compound B1 may, in various embodiments, have a pKₐ at about 25° C. ranging from about 7 to about 9, for instance, about 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9, including all ranges and subranges therebetween. The at least one buffer compound B1 can be included in the suspension buffer S1 in an amount sufficient to adjust the pH to a value ranging from about 7 to about 10, such as from about 7.5 to about 9.5, from about 8.5 to about 9, or from about 8 to about 8.3, including all ranges and subranges therebetween. For example, S1 can have a pH equal to about 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10, including all ranges and subranges therebetween.

After suspension, a lysis buffer S2 can be added to form a lysate from the suspended sample (see, e.g., step L in FIG. 1). The lysis buffer S2 can comprise, in various embodiments, at least one detergent chaotropic agent (DC) and at least one buffer compound (B2). The suspension and the lysis buffer S2 can be combined in any manner known in the art, for example, the lysis buffer S2 can be added to the suspension and mixed, e.g., by inversion. In certain embodiments, the mixture may be inverted multiple times, such as at least five times, at least ten times, or more. Combinations by inversion can ensure optimal cell lysis efficiency and/or final nucleic acid yield. Additionally, the suspension can optionally be incubated in the lysis buffer S2 for a period of time sufficient to lyse the sample. This time period can range, for example, from about 30 seconds to about 10 minutes, such as from about 1 minute to about 8 minutes, from about 2 minutes to about 5 minutes, or from about 3 minutes to about 4 minutes, including all ranges and subranges therebetween.

The at least one detergent chaotropic agent DC can be present in the lysis buffer S2 in a concentration ranging, for example, from about 1% to about 10% by weight, such as from about 2% to about 9%, from about 3% to about 8%, from about 4% to about 7%, or from about 5% to about 6%, including all ranges and subranges therebetween. For example, the DC concentration can be about 1%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, or 10% by weight, including all ranges and subranges therebetween. According to various embodiments, the at least one detergent chaotropic agent DC can be chosen from cationic, anionic, nonionic, and zwitterionic detergents or surfactants, such as sodium dodecyl sulfate (SDS), sodium dodecylbenzene sulfonate (SDBS), ammonium lauryl sulfate (ALS), potassium dodecyl sulfate (PDS), sodium myreth sulfate, octylphenol ethoxylates (e.g., Triton™ X-100 or X-114), polyoxyethylene sorbitan monolaureates (e.g., Tween® 20, 40, or 80), and combinations thereof. In certain non-limiting embodiments, the at least one detergent chaotropic agent DC can be chosen from SDS and SDBS. The at least one detergent chaotropic agent DC may, in various embodiments, be used to disrupt cell membranes and/or denature lipids and/or proteins in the sample.

The at least one buffer compound B2 can be present in the lysis buffer S2 in a concentration greater than or equal to about 0.2 M, such as greater than or equal to about 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M, 1 M, or higher, including all ranges and subranges therebetween. According to various embodiments, the at least one buffer compound B2 can be chosen from hydroxides such as sodium hydroxide (NaOH), lithium hydroxide (LiOH), potassium hydroxide (KOH), rubidium hydroxide (RbOH), cesium hydroxide (CsOH), and combinations thereof. In certain non-limiting embodiments, the at least one buffer compound B2 can be NaOH.

The at least one buffer compound B2 may, in various embodiments, have a pK_{b} at about 25° C. ranging from about 0.1 to about 2, for instance, about 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2, including all ranges and subranges therebetween. The at least one buffer compound B2 can be included in the lysis buffer S2 in an amount sufficient to adjust the pH to a value ranging from about 10 to about 13, such as from about 10.5 to about 12.5, or from about 11 to about 12, including all ranges and subranges therebetween. For example, S2 can have a pH equal to about 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, or 13, including all ranges and subranges therebetween.

After lysis, a binding (or neutralization) buffer (S3) can be added to the lysate to form a solution. The binding buffer S3 can comprise at least one chaotropic agent (C1), at least one alcohol (A1), optionally at least one salt (Z1), and at least one buffer compound (B3). The lysate and the binding buffer S3 can be combined in any manner known in the art, for example, the binding buffer S3 can be added to the lysate and mixed, e.g., by inversion. In certain embodiments, the mixture may be inverted multiple times, such as at least five times, at least ten times, or more. The lysate can optionally be incubated in the binding buffer S3 for a period of time sufficient to neutralize the sample and/or promote aggregate formation of unwanted contaminants (which can appear as cloudy particulates). This time period can range, for example, from about 1 minute to about 30 minutes, such as from about 2 minutes to about 25 minutes, from about 3 minutes to about 20 minutes, from about 4 minutes to about 15 minutes, or from about 5 minutes to about 10 minutes, including all ranges and subranges therebetween.

The at least one chaotropic agent C1 can be present in the binding buffer S3 in a concentration ranging, for example, from about 4 M to about 6 M, such as from about 4.2 M to about 5.8 M, from about 4.4 M to about 5.6 M, from about 4.6 M to about 5.4 M, or from about 4.8 M to about 5.2 M, including all ranges and subranges therebetween. For example, the C1 concentration can be about 4 M, 4.1 M, 4.2 M, 4.3 M, 4.4 M, 4.5 M, 4.6 M, 4.7 M, 4.8 M, 4.9 M, 5 M, 5.1 M, 5.2 M, 5.3 M, 5.4 M, 5.5 M, 5.6 M, 5.7 M, 5.8 M, 5.9 M, or 6 M, including all ranges and subranges therebetween. According to various embodiments, the at least one chaotropic agent C1 can be chosen from guanidine hydrochloride (GuHCl), guanidium thiocyanate (GuSCN), urea, and combinations thereof. In certain non-limiting embodiments, the at least one chaotropic agent C1 can be chosen from GuHCl and GuSCN. The at least one chaotropic agent C1 may, in various embodiments, be used to disrupt cell membranes and/or denature lipids and/or proteins in the sample.

The at least one alcohol A1 can be present in the binding buffer S3 in a concentration ranging, for example, from about 1% to about 5% by volume, such as from about 2% to 4%, or from about 2.5% to about 3% by volume, including all ranges and subranges therebetwen. For example, the A1 concentration can be about 1%, 1.5%, 2%, 3%, 3.5%, 4%, 4.5%, or 5% by volume, including all ranges and subranges therebetween. According to various embodiments, the at least one alcohol A1 can be chosen from isopropanol, ethanol, methanol, butanol, and combinations thereof. In certain non-limiting embodiments, the at least one alcohol A1 can be isopropanol. The at least one alcohol A1 may, in various embodiments, be chaotropic and may be used to disrupt cell membranes and/or denature proteins in the sample.

The at least one salt Z1, if present, can be present in the binding buffer S3 in a concentration ranging, for example, from about 0.2 M to about 2 M, such as from about 0.4 M to about 1.8 M, from about 0.6 M to about 1.6 M, from about 0.8 M to about 1.4 M, or from about 1 M to about 1.2 M, including all ranges and subranges therebetween. For example, the Z1 concentration can be about 0.2 M, 0.3 M, 0.4 M, 0.5 M, 0.6 M, 0.7 M, 0.8 M, 0.9 M, 1 M, 1.1 M, 1.2 M, 1.3 M, 1.4 M, 1.5 M, 1.6 M, 1.7 M, 1.8 M, 1.9 M, or 2 M, including all ranges and subranges therebetween. According to various embodiments, the at least one salt Z1 can be chosen from ammonium sulfate ((NH₄)₂SO₄), ammonium acetate (NH₄Ac), lithium acetate (LiAc), potassium acetate (KAc), sodium acetate (NaAc), sodium chloride (NaCl), and combinations thereof. In certain non-limiting embodiments, the at least one salt Z1 can be ammonium sulfate. The at least one salt Z1 can, in various embodiments, be included to improve nucleic acid purification or can, in other embodiments, be excluded, although this may produce lower yields. Increased salt concentration in the binding buffer S3 can, for example, promote binding of the nucleic acid to the magnetic particles and/or precipitation of unwanted contaminant aggregates.

The at least one buffer compound B3 can be present in the binding buffer S3 in a concentration ranging from about 0.25% to about 3% by weight, such as from about 0.5% to about 2.5%, or from about 1% to about 1.5%, including all ranges and subranges therebetween. For example, the B3 concentration can be about 0.25%, 0.5%, 0.75%, 1%, 1.25%, 1.5%, 1.75%, 2%, 2.25%, 2.5%, 2.75%, or 3% by weight, including all ranges and subranges therebetween. According to various embodiments, the at least one buffer compound B3 can be chosen from glacial acetic acid, hydrochloric acid, and combinations thereof.

The at least one buffer compound B3 may, in various embodiments, have a pKₐ at about 25° C. ranging from about 4 to about 7, for instance, about 4, 4.25, 4.5, 4.75, 5, 5.25, 5.5, 5.75, 6, 6.25, 6.5, 6.75, or 7, including all ranges and subranges therebetween. The at least one buffer compound B3 can be included in the binding buffer S3 in an amount sufficient to adjust the pH to a value ranging from about 4 to about 9, such as from about 5 to about 8, or from about 6 to about 7, including all ranges and subranges therebetween. For example, S3 can have a pH equal to about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9, including all ranges and subranges therebetween.

According to various embodiments, alkaline conditions (e.g., pH>10), e.g., produced by the addition of the lysis buffer S2 can be used to denature both pDNA and genomic DNA present in the sample. Subsequent neutralization with the binding buffer S3 can contribute to the overall effectiveness of the disclosed methods, particularly for pDNA purification, in a variety of ways. For instance, neutralization can cause the genomic DNA to base-pair in an intrastrand manner, thus forming an insoluble aggregate that can precipitate out of solution. By contrast, it is believed that the covalently closed nature of the circular pDNA can promote interstrand rehybridization, thus allowing the pDNA to remain in solution. In addition, when the binding buffer S3 comprises at least one salt Z1, such as potassium, sodium, or lithium salts, these salts can react with one or more components in solution to form additional precipitates. For example, the potassium salt of SDS is insoluble. Thus, the precipitation and aggregation of both protein(s) and detergent(s) can assist in the entrapment of high-molecular weight genomic DNA. These aggregates and any macromolecules can then be removed from solution, e.g., by centrifugation or other known methods such as filtration (see, e.g., step C in FIG. 1), which is discussed in more detail below.

After addition of the binding buffer, the solution can be combined with at least one magnetic particle to produce a combined solution (see, e.g., step B in FIG. 1). As used herein the term “magnetic particle” and variations thereof is intended to denote a particle with a magnetic, e.g., paramagnetic or superparamagnetic, core coated with at least one material having a surface to which nucleic acid can reversibly bind. Suitable magnetic particles can include, for example, carboxyl coated paramagnetic particles, silica-based paramagnetic particles, and the like. Silica-based magnetic particles can comprise, in some embodiments, a paramagnetic core coated with siliceous oxide, thus providing a hydrous siliceous oxide adsorptive surface to which nucleic acid can bind (e.g., a surface comprising silanol groups). The magnetic particles can, in additional embodiments, be surface-modified to produce functionalized surfaces, such as weakly or strongly positively charged, weakly or strongly negatively charged, or hydrophobic surfaces, to name a few.

Non-limiting examples of commercially available magnetic particles include Qbeads from MagQu Co. Ltd., Grace beads from W. R. Grace & Co., and the like. The magnetic particles can have any size suitable for binding nucleic acid, including commercially available sizes, such as a diameter ranging from about 0.3 μm to about 10 μm in diameter, e.g., about 0.3, 0.5, 1, 2, 3, 4, 5, 6, 7, 8, 9, or 10 μm in diameter, including all ranges and subranges therebetween. Qbeads can, for example, have an average diameter ranging from about 4 μm to about 5 μm, and Grace beads can have an average diameter ranging from about 5 μm to about 10 μm.

The magnetic particles can, in some embodiments, be added to the solution as a suspension in at least one medium. The medium can be chosen, for example, from water and/or chaotropic agents. In certain embodiments, the magnetic particle can be Qbeads suspended in water or Grace beads suspended in a chaotropic agent such as GuSCN. The concentration of the beads in the suspension may vary as desired and can range, for instance, from about 10 μg/ml to about 100 μg/ml, such as from about 20 μg/ml to about 90 μg/ml, from about 30 μg/ml to about 80 μg/ml, from about 40 μg/ml to about 70 μg/ml, or from about 50 μg/ml to about 60 μg/ml, including all ranges and subranges therebetween. The concentration of the chaotropic agent in the suspension, if used, can also vary as desired and may range, for example, from about 1 M to about 8 M, such as from about 2 M to about 6 M, or from about 4 M to about 5 M, including all ranges and subranges therebetween. The pH of the solution comprising the magnetic beads can, for example, range from about 4 to about 9, such as from about 5 to about 8, or from about 6 to about 7, including all ranges and subranges therebetwen.

Without wishing to be bound by theory, it is believed that the relatively high concentration of chaotropic agent(s) and/or salt(s) introduced by the binding buffer S3 can enhance the ability of nucleic acid, such as pDNA, to reversibly (e.g., non-covalently) bind to the surface of the magnetic particle, such as a silica surface. The magnetic particles thus modified, e.g., comprising reversibly bound nucleic acid, can then be separated from the unbound contaminants. For instance, a magnet can be placed in proximity to the modified magnetic particles and used to draw the particles together, e.g., to form an aggregate or pellet. In certain embodiments, a container, such as a tube, containing a combined solution comprising the modified magnetic particles, can be placed on a magnetic stand, which can gather and somewhat immobilize the particles while the remaining solution is removed.

After binding the nucleic acid to the magnetic particles and after separation of the modified magnetic particles using, e.g., a magnet, the particles can then be combined, rinsed, or washed with one or more wash buffers (see, e.g., step W in FIG. 1). A first wash buffer (W1) can comprise, for example, at least one chaotropic agent (C2), at least one ion chelating agent (IC2), at least one alcohol (A2), and at least one buffer compound (B4).

The at least one chaotropic agent C2 can be present in the first wash buffer W1 in a concentration ranging, for example, from about 4 M to about 6 M, such as from about 4.2 M to about 5.8 M, from about 4.4 M to about 5.6 M, from about 4.6 M to about 5.4 M, or from about 4.8 M to about 5 M, including all ranges and subranges therebetwen. For example, the C2 concentration can be about 4 M, 4.1 M, 4.2 M, 4.3 M, 4.4 M, 4.5 M, 4.6 M, 4.7 M, 4.8 M, 4.9 M, 5 M, 5.1 M, 5.2 M, 5.3 M, 5.4 M, 5.5 M, 5.6 M, 5.7 M, 5.8 M, 5.9 M, or 6 M, including all ranges and subranges therebetween. According to various embodiments, the at least one chaotropic agent C2 can be chosen from guanidine hydrochloride (GuHCl), guanidium thiocyanate (GuSCN), urea, and combinations thereof. In certain non-limiting embodiments, the at least one chaotropic agent C2 can be chosen from GuHCl and GuSCN. The at least one chaotropic agent C2 may, in various embodiments, be used to promote nucleic acid binding to the magnetic particle surface.

The at least one ion chelating agent IC2 can be present in the first wash buffer W1 in a concentration ranging, for example, from about 1 mM to about 10 mM, such as from about 2 mM to about 9 mM, from about 3 mM to about 8 mM, from about 4 mM to about 7 mM, or from about 5 mM to about 6 mM, including all ranges and subranges therebetween. For example, the IC2 concentration can be about 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, or 10 mM, including all ranges and subranges therebetween. According to various embodiments, the at least one ion chelating agent IC2 can be chosen from ethylenediamine tetraacetic acid (EDTA) and isomers thereof such as ethylenediamine-N,N′-disuccinic acid (EDDS); cyclohexane-1,2-diaminetetraacetic acid (CDTA); iminodisuccinic acid (IDS); polyaspartic acid (PASA); methylglycinediacetic acid (MGDA); L-glutamic acid N,N-diacetic acid, tetrasodium salt (GLDA), and combinations thereof. In certain non-limiting embodiments, the at least one ion chelating agent IC2 can be chosen from EDTA and isomers thereof. The at least one ion chelating agent IC2 may, in various embodiments, serve to reduce oxidation damage and/or contaminating nuclease activity and/or to chelate metal ions such as magnesium.

The at least one alcohol A2 can be present in the first wash buffer W1 in a concentration ranging, for example, from about 30% to about 50% by volume, such as from about 35% to about 45% by volume, including all ranges and subranges therebetween. For example, the A2 concentration can be about 30%, 31%, 32%, 33%, 34%, 35%, 36%, 37%, 38%, 39%, 40%, 41%, 42%, 43%, 44%, 45%, 46%, 47%, 48%, 49%, or 50%, including all ranges and subranges therebetween. According to various embodiments, the at least one alcohol A2 can be chosen from isopropanol, ethanol, methanol, butanol, and combinations thereof. In certain non-limiting embodiments, the at least one alcohol A2 can be isopropanol.

The at least one buffer compound B4 can be present in the first wash buffer W1 in a concentration ranging, for instance, from about 10 mM to about 100 mM, such as from about 20 mM to about 90 mM, from about 30 mM to about 80 mM, from about 40 mM to about 70 mM, or from about 50 mM to about 60 mM, including all ranges and subranges therebetween. For example, the B4 concentration can be about 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM, including all ranges and subranges therebetween. According to various embodiments, the at least one buffer compound B4 can be chosen from Tris, Tris-HCl, 3-(N-morpholino)propanesulfonic acid (MOPS), 4-(2-hydroxyethyl)-1-piperazineethanesulfonic acid (HEPES), 2-(N-morpholino)ethanesulfonic acid (MES), and combinations thereof. In certain non-limiting embodiments, the at least one buffer compound B4 can be chosen from Tris and Tris-HCl.

The at least one buffer compound B4 may, in various embodiments, have a pKₐ at about 25° C. ranging from about 7 to about 9, for instance, about 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9, including all ranges and subranges therebetween. The at least one buffer compound B4 can be included in the first wash buffer W1 in an amount sufficient to adjust the pH to a value ranging from about 6 to about 8, such as from about 6.5 to about 7.5, or from about 6.8 to about 7.2, including all ranges and subranges therebetween. For example, W1 can have a pH equal to about 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8, including all ranges and subranges therebetween.

The modified magnetic particles can further be combined, rinsed, or washed with a second wash buffer (W2), which may comprise at least one alcohol (A3) and optionally at least one salt (Z2). The at least one alcohol A3 can be present in the second wash buffer W2 in a concentration ranging, for example, from about 70% to 100% by volume, such as from about 75% to about 95%, or from about 80% to about 90% by volume, including all ranges and subranges therebetween. For example, the A3 concentration can be about 70%, 75%, 80%, 85%, 90%, 95%, or 100%, including all ranges and subranges therebetween. According to various embodiments, the at least one alcohol A3 can be chosen from isopropanol, ethanol, methanol, butanol, and combinations thereof. In certain non-limiting embodiments, the at least one alcohol A3 can be ethanol.

The at least one salt Z2, if present, can be present in the second wash buffer W2 in a concentration ranging, for example, from about 10 mM to about 100 mM, such as from about 20 mM to about 90 mM, from about 30 mM to about 80 mM, from about 40 mM to about 70 mM, or from about 50 mM to about 60 mM, including all ranges and subranges therebetween. For example, the Z2 concentration can be about 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM, including all ranges and subranges therebetween. According to various embodiments, the at least one salt Z2 can be chosen from ammonium sulfate ((NH₄)₂SO₄), ammonium acetate (NH₄Ac), lithium acetate (LiAc), potassium acetate (KAc), sodium acetate (NaAc), sodium chloride (NaCl), and combinations thereof. In certain non-limiting embodiments, the at least one salt Z2 can be chosen from NaAC and NH₄AC.

According to various embodiments, the pH of the second wash buffer W2 can range, for example, from about 6 to about 8, such as from about 6.2 to about 7.5, from about 6.5 to about 7, or from about 6.4 to about 6.8, including all ranges and subranges therebetween. The pH of the second wash buffer W2 can be adjusted by varying the amount of alcohol and/or salt, or can be adjusted using one or more buffer compounds, e.g., glacial acetic acid or NaOH, as disclosed herein.

After the addition and removal of the wash buffers W1 and W2, modified magnetic particles with nucleic acid reversibly bound to the surface may be provided, which can be free or substantially free of contaminants such as cell debris, lipids, proteins, and/or unwanted nucleic acids. According to various embodiments, the modified magnetic particles thus produced can then be combined with an elution buffer (E1) to release the bound nucleic acid and separate it from the magnetic particles (see, e.g., step E in FIG. 1). The modified magnetic particles can be incubated in the elution buffer E1 for a period of time sufficient to release the nucleic acids, such as from about 30 seconds to about 10 minutes, for example, from about 45 seconds to about 9 minutes, from about 1 minute to about 8 minutes, from about 2 minutes to about 7 minutes, from about 3 minutes to about 6 minutes, or from about 4 minutes to about 5 minutes, including all ranges and subranges therebetween.

The elution buffer E1 can comprise, for example, water or a relatively low salt solution comprising, for instance, at least one buffer compound (e.g., Tris and the like), at least one salt, and/or at least one ion chelating agent (e.g., EDTA and the like). In certain embodiments, the elution buffer E1 can comprise from about 10 mM to about 100 mM of at least one buffer compound and from about 0.1 mM to about 10 mM of at least one ion chelating agent. According to one non-limiting embodiment, the elution buffer E1 can comprise 10 mM Tris and 1 mM EDTA.

The magnetic particles (no longer attached to the nucleic acid) can subsequently be removed from the solution, e.g., separated using a magnet, yielding a purified nucleic acid in solution as the final product (see, e.g., step P in FIG. 1). For example, the methods and kits disclosed herein can be used to provide a purified pDNA product. According to various embodiments, the methods and kits disclosed herein can be used to efficiently produce transfection-grade pDNA in a short time period. For instance, in non-limiting embodiments, the methods disclosed herein can be carried out in a time period of less than about 30 minutes, such as less than about 20 minutes. The methods disclosed herein can, in certain embodiments, produce 10 μg of pDNA on average from 1 ml of overnight bacterial culture (O.D. 600≈1) in approximately 20 minutes.

It is to be understood that the components of the various buffer solutions can, in some embodiments, be used interchangeably, e.g., can be the same or different from each other. For instance, chaotropic agents C1 and C2 can be identical or different. Similarly, ion chelating agents IC1 and IC2; salts Z1 and Z2; alcohols A1, A2, and A3; and buffers B1, B2, B3, and B4; respectively, can be identical or different. Likewise, the concentrations of these components can vary and can, in some instances be identical or similar, depending on the desired application.

It is also to be understood that the methods disclosed herein can further comprise additional steps known in the art, such as centrifugation, filtration, and the like. By way of non-limiting example, after addition of the binding buffer S3, the resulting solution can be centrifuged or filtered to remove unwanted agglomerates or macromolecules. Centrifugation of the sample and/or magnetic particles can also be optionally carried out according to various embodiments. In such instances, centrifugation can be carried out at an acceleration ranging from about 10,000 g to about 18,000 g, such as from about 12,000 g to about 16,000 g, or from about 14,000 g to about 5,000 g, including all ranges and subranges therebetween. Centrifugation can proceed, in various embodiments, for a time period ranging from about 30 seconds to about 15 minutes, for example, from about 1 minute to about 14 minutes, from about 2 minutes to about 13 minutes, from about 3 minutes to about 12 minutes, from about 4 minutes to about 11 minutes, from about 5 minutes to about 10 minutes, from about 6 minutes to about 9 minutes, or from about 7 minutes to about 8 minutes, including all ranges and subranges therebetween.

In other non-limiting embodiments, the methods disclosed herein do not include any centrifugation steps, which may enhance the ability to automate the process. Other optional steps can include air drying, e.g., after rinsing the modified magnetic particles with wash buffers W1 and W2, the particles may be air dried for a period of time ranging from about 1 minute to about 10 minutes, such as from about 2 minutes to about 9 minutes, from about 3 minutes to about 8 minutes, from about 4 minutes to about 7 minutes, or from about 5 minutes to about 6 minutes, including all ranges and subranges therebetween. Removal and/or transfer of the various samples, solutions, or portions of the samples or solutions to new containers, such as tubes, can also be carried out during the methods disclosed herein as desired or necessary.

### Kits

The disclosure also relates to kits for nucleic acid purification, the kits comprising a suspension buffer, a lysis buffer, a binding/neutralization buffer, at least one magnetic particle, a first wash buffer, a second wash buffer, and optionally an elution buffer. The buffers can correspond, in various embodiments, to the buffers S1, S2, S3, B1, W1, W2, and E1, as disclosed above with reference to the purification methods. It should be understood that the various embodiments discussed above with respect to each of the buffers can be combined in any manner and without limitation to form the kits disclosed herein.

According to various embodiments, each buffer can be supplied in the kit at with predetermined concentrations for each component that are ready-to-use. Alternatively, one or more concentrated solutions can be provided, to be diluted by the end user with the appropriate type and amount of solvent to produce the ready-to-use buffers. For example, in certain embodiments, a concentrated wash buffer W2 can be provided, which can be diluted by the user with an alcohol, such as ethanol, e.g., to a final concentration of 70% or greater by volume of ethanol.

The kit can, in some embodiments, further include instructions to the end user regarding the purification protocol and/or any dilution instructions. According to other embodiments, the kit can further comprise various additional components or equipment, such as a magnetic stand, tubes, centrifuge, media and/or antibiotics for bacterial culture, solvents, and/or RNase.

It will be appreciated that the various disclosed embodiments may involve particular features, elements or steps that are described in connection with that particular embodiment. It will also be appreciated that a particular feature, element or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “a buffer” includes examples having two or more such “buffers” unless the context clearly indicates otherwise.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Other than in the Examples, all numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a concentration greater than 2 M” and “a concentration greater than about 2 M” both include embodiments of “a concentration greater than about 2 M” as well as “a concentration greater than 2 M.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a buffer that comprises A+B+C include embodiments where a buffer consists of A+B+C, and embodiments where a buffer consists essentially of A+B+C.

It will be apparent to those skilled in the art that various modifications and variations can be made to the present disclosure without departing from the spirit and scope of the disclosure. Since modifications combinations, sub-combinations and variations of the disclosed embodiments incorporating the spirit and substance of the disclosure may occur to persons skilled in the art, the disclosure should be construed to include everything within the scope of the appended claims and their equivalents.

The following Examples are intended to be non-restrictive and illustrative only, with the scope of the invention being defined by the claims.

### EXAMPLES

### Exemplary Purification Protocol

For purposes of discussion only, an exemplary protocol for purifying pDNA from a bacterial culture is provided below. Of course, this protocol is not intended to limit and should not be construed as limiting on the appended claims.
a) bacterial culture sample preparation: prepare 5 ml of overnight bacterial culture, O.D. 600≈1, with an appropriate medium and antibiotic;
b) suspension buffer preparation: add RNase to suspension buffer S1 to a final concentration of 100 μg/ml;
c) suspension of sample: transfer 1 ml of overnight bacterial culture to a 1.5 microfuge tube and harvest cells by centrifugation at 12,000 g for 1 minute followed by culture medium removal and resuspension with 250 μl of prepared S1 buffer;
d) lysis of sample: add 250 μl of lysis buffer S2 to the suspension and mix by inversion a minimum of 10 times;
e) preparation for binding: add 350 μl of binding buffer S3 to cell lysate and mix by inversion a minimum of 10 times, or until a cloudy particulate is visible;
f) cell lysate clearing: centrifuge the solution at 12,000 g for 10 minutes and transfer the cleared lysate to a new microfuge tube;
g) preparation of magnetic particles: vortex a suspension of magnetic particles in water or chaotropic agent for at least 1 minute before use;
h) pDNA binding: add 50 μl magnetic particle solution to the cleared lysate and mix by inversion followed by an incubation period of 5 minutes at room temperature;
i) separation of pDNA-bound magnetic particles: magnetically separate magnetic particles with reversibly bound pDNA using a magnetic stand for 1 minute and remove remaining lysate;
j) washing of pDNA-bound magnetic particles: wash magnetic particles comprising reversibly bound pDNA once with 500 μl of wash buffer W1 and once with 700 μl of wash buffer W2;
k) removal of wash buffer: remove wash buffers W1 and W2 and allow magnetic particles with reversibly bound pDNA to air dry upside down on the magnetic stand for 5 minutes;
l) elution of pDNA: remove tube from magnetic stand and add 100 μl (or 50 μl to produce a more concentrated sample) of water and incubate for 1 minute at room temperature;
m) purification of pDNA: place tube on a magnetic stand for 1 minute to remove magnetic particles and transfer eluted pDNA to a new microfuge tube.

### Comparative Example 1

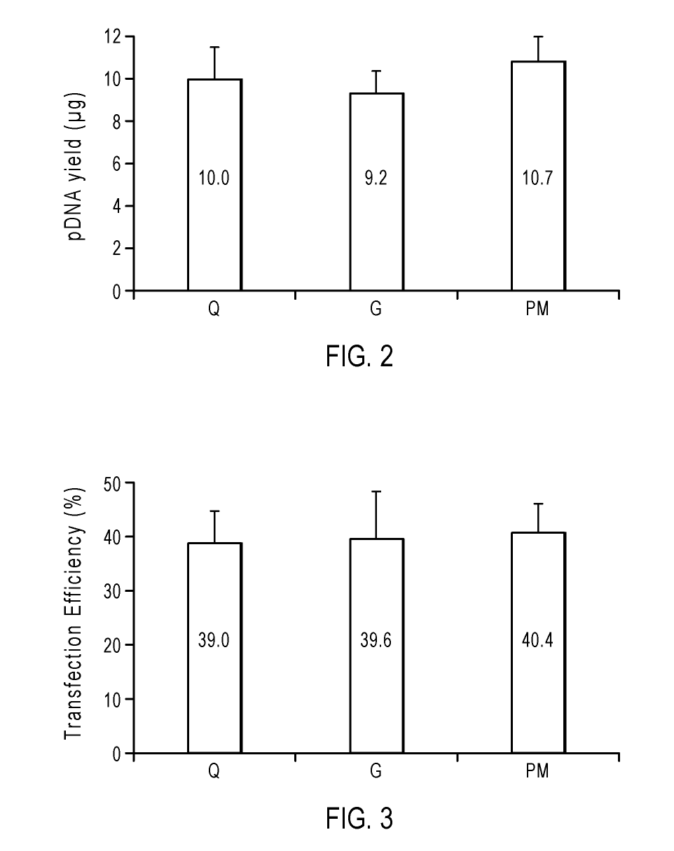

pDNA was purified from bacterial cultures using the above protocol with 1000 μg Qbeads or 5000 μg Grace beads. The same bacterial culture was also purified using the Wizard MagneSil Tfx™ system by Promega. The average total pDNA yield for each method was quantified and is presented in FIG. 2. The average total pDNA yield for the inventive method employing Qbeads was 10.0 μg from a 1 ml sample of bacterial culture (O.D. 600≈1). The average pDNA yield for the inventive method employing Grace beads was 9.2 μg, whereas the comparative Promega method yielded 10.7 μg pDNA.

Transfection efficiency for each pDNA sample was then determined by transfecting eGFP-encoded plasmid, pEGFP-C1, into Hela cells. Transfected cells were fixed with 4% paraformaldehyde for 15 minutes and stained with DAPI for nuclei visualization. Fluorescent images were taken with appropriate filters for GFP and nuclei followed by ImageJ image analyses by first converting binary images (black and white) and counting criteria defined to count GFP positive cells and total cells (nuclei). Transfection efficiency was determined by dividing GFP positive cells by the total number of cells in the viewing field. Transfection efficiency for each method is illustrated in FIG. 3, where the error bar represents standard deviations of at least 8 images. The transfection efficiencies were 39.0%, 39.6%, and 40.4%, for methods employing Qbeads, Grace beads, and the Promega kit, respectively.

Accordingly, FIGS. 2-3 demonstrate that the inventive methods, which were carried out in about 20 minutes, provide pDNA yield and quality comparable to that of the benchmark comparative Promega kit.

### Comparative Example 2

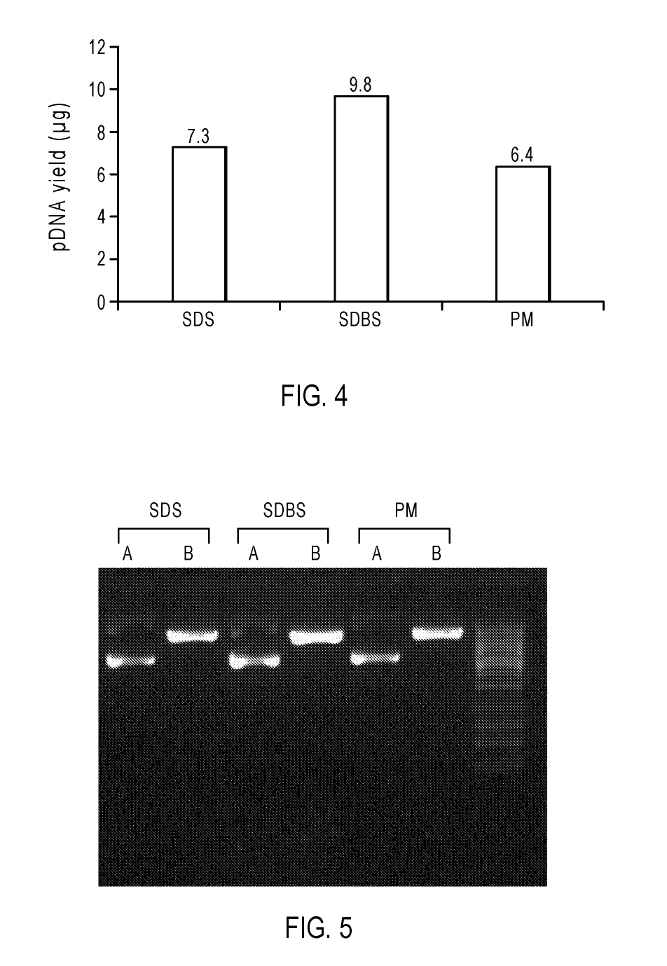

pDNA was purified from bacterial cultures using the above protocol with lysis buffers S2 comprising SDS or SDBS. The same bacterial culture was also purified using the Wizard MagneSil Tfx™ system by Promega. The average total pDNA yield for each method was quantified and is presented in FIG. 4. The average total pDNA yield for the inventive method employing SDS in the lysis buffer S2 was 7.3 μg from a 1 ml sample of bacterial culture (O.D. 600≈1). The average pDNA yield for the inventive method employing SDBS in the lysis buffer S2 was 9.8 μg, whereas the comparative Promega method yielded 6.4 μg pDNA.

Downstream applications, such as restriction digest and transfection were performed to determine the quality of the purified pDNA produced by each method. For restriction digest, the restriction enzyme EcoRI was used to linearize the pDNA. FIG. 5 demonstrates the agarose gel electrophoresis analysis of the pDNA samples (uncut A or cut with EcoRI B) for the three different methods. As demonstrated by FIG. 5, the pDNA produced by each method could be digested by the restriction enzyme EcoRI.

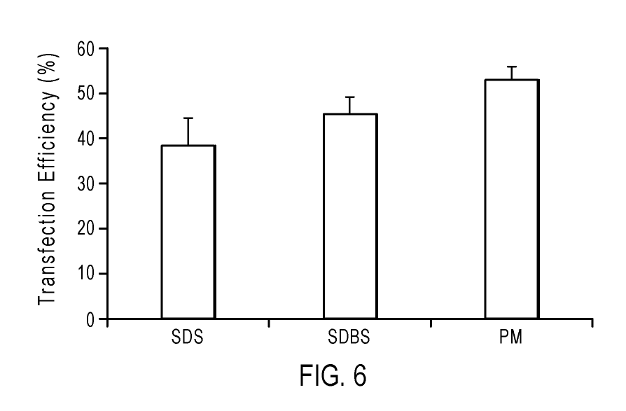

Transfection efficiency for each pDNA sample was then determined by transfecting eGFP-encoded plasmid, pEGFP-C1, into Hela cells. Transfected cells were fixed with 4% paraformaldehyde for 15 minutes and stained with DAPI for nuclei visualization. Fluorescent images were taken with appropriate filters for GFP and nuclei followed by ImageJ image analyses by first converting binary images (black and white) and counting criteria defined to count GFP positive cells and total cells (nuclei). Transfection efficiency was determined by dividing GFP positive cells by the total number of cells in the viewing field. Transfection efficiency for each method is illustrated in FIG. 6, where the error bar represents standard deviations of at least 8 images. The transfection efficiencies were 38.0%, 45.4%, and 52.9%, for methods employing SDS lysis buffer, SDBS lysis buffer, and the Promega kit, respectively.

Accordingly, FIGS. 4-6 demonstrate that the inventive methods, which were carried out in about 20 minutes, provide pDNA yield and/or quality that is improved over or comparable to that of the benchmark comparative Promega kit.

1. A method for nucleic acid purification, comprising:
(a) combining a sample comprising at least one nucleic acid with a suspension buffer to form a suspension;
(b) combining the suspension with a lysis buffer to form a lysate;
(c) combining the lystate with a binding buffer to form a solution;
(d) combining the solution with at least one magnetic particle to form a combined solution comprising at least one modified magnetic particle, wherein the at least one modified magnetic particle is reversibly bound to the at least one nucleic acid;
(e) separating the at least one modified magnetic particle from the combined solution;
(f) washing the at least one modified magnetic particle with a first wash buffer and a second wash buffer; and
(g) combining the at least one modified magnetic particle with an elution buffer;
wherein:
the suspension buffer comprises at least one first ion chelating agent, present in a concentration ranging from about 1 mM to about 10 mM, and at least one first buffer compound, present in a concentration ranging from about 10 mM to about 100 mM;
the lysis buffer comprises at least one detergent chaotropic agent, present in a concentration ranging from about 1% to about 10% by weight, and at least one second buffer compound, present in a concentration greater than or equal to about 0.2 M;
the binding buffer comprises at least one first chaotropic agent, present in a concentration ranging from about 4 M to about 6 M, optionally at least one first salt, present in a concentration ranging from about 0.1 M to about 2 M, at least one first alcohol, present in a concentration ranging from about 1% to about 5% by volume, and at least one third buffer compound, present in a concentration ranging from about 0.25% to about 3% by weight;
the first wash buffer comprises at least one second chaotropic agent, present in a concentration ranging from about 4 M to about 6 M, at least one second ion chelating agent, present in an concentration ranging from about 1 mM to about 10 mM, at least one second alcohol, present in a concentration ranging from about 30% to about 50% by volume, and at least one fourth buffer compound, present in a concentration ranging from about 10 mM to about 100 mM; and
the second wash buffer comprises at least one third alcohol and optionally at least one second salt.

2. The method of claim 1, wherein the sample comprising the at least one nucleic acid is at least one bacterial cell comprising plasmid DNA.

3. The method of claim 1, wherein the at least one first ion chelating agent is chosen from ethylenediamine tetraacetic acid (EDTA); ethylenediamine-N,N′-disuccinic acid (EDDS); cyclohexane-1,2-diaminetetraacetic acid (CDTA); iminodisuccinic acid (IDS); polyaspartic acid (PASA); methylglycinediacetic acid (MGDA); L-glutamic acid N,N-diacetic acid, tetrasodium salt (GLDA), and combinations thereof, and
wherein the at least one first buffer compound is chosen from Tris, Tris-HCl, 3-(N-morpholino)propanesulfonic acid (MOPS), 4-(2-hydroxyethyl)-1-piperazineethanesulfonic acid (HEPES), 2-(N-morpholino)ethanesulfonic acid (MES), and combinations thereof.

4. The method of claim 1, wherein the at least one detergent chaotropic agent is chosen from sodium dodecyl sulfate (SDS), sodium dodecylbenzene sulfonate (SDBS), ammonium lauryl sulfate (ALS), potassium dodecyl sulfate (PDS), sodium myreth sulfate, octylphenol ethoxylates, polyoxyethylene sorbitan monolaureates, and combinations thereof, and
wherein the at least one second buffer compound is chosen from sodium hydroxide (NaOH), lithium hydroxide (LiOH), potassium hydroxide (KOH), rubidium hydroxide (RbOH), cesium hydroxide (CsOH), and combinations thereof.

5. The method of claim 1, wherein the at least one first chaotropic agent is chosen from guanidine hydrochloride (GuHCl), guanidium thiocyanate (GuSCN), urea, and combinations thereof,
wherein the at least one first salt is chosen from ammonium sulfate ((NH₄)₂SO₄), ammonium acetate (NH₄Ac), lithium acetate (LiAc), potassium acetate (KAc), sodium acetate (NaAc), sodium chloride (NaCl), and combinations thereof,
wherein the at least one first alcohol is chosen from isopropanol, ethanol, methanol, butanol, and combinations thereof, and
wherein the at least one third buffer compound is chosen from glacial acetic acid, hydrochloric acid, and combinations thereof.

6. The method of claim 1, wherein the at least one second chaotropic agent is chosen from guanidine hydrochloride (GuHCl), guanidium thiocyanate (GuSCN), urea, and combinations thereof,
wherein the at least one second ion chelating agent is chosen from ethylenediamine tetraacetic acid (EDTA); ethylenediamine-N,N′-disuccinic acid (EDDS); cyclohexane-1,2-diaminetetraacetic acid (CDTA); iminodisuccinic acid (IDS); polyaspartic acid (PASA); methylglycinediacetic acid (MGDA); L-glutamic acid N,N-diacetic acid, tetrasodium salt (GLDA), and combinations thereof,
wherein the at least one second alcohol is chosen from isopropanol, ethanol, methanol, butanol, and combinations thereof, and
wherein the at least one fourth buffer compound is chosen from Tris, Tris-HCl, 3-(N-morpholino)propanesulfonic acid (MOPS), 4-(2-hydroxyethyl)-1-piperazineethanesulfonic acid (HEPES), 2-(N-morpholino)ethanesulfonic acid (MES), and combinations thereof.

7. The method of claim 1, wherein the at least one third alcohol is chosen from isopropanol, ethanol, methanol, butanol, and combinations thereof, and
wherein the at least one second salt is chosen from ammonium sulfate ((NH₄)₂SO₄), ammonium acetate (NH₄Ac), lithium acetate (LiAc), potassium acetate (KAc), sodium acetate (NaAc), sodium chloride (NaCl), and combinations thereof.

8. The method of claim 1, wherein the at least one magnetic particle is chosen from carboxyl coated paramagnetic particles, silica-based paramagnetic particles, and combinations thereof.

9. The method of claim 1, further comprising centrifuging the solution to remove at least one agglomerate or macromolecule prior to (d) combining the solution with the at least one magnetic particle.

10. The method of claim 1, further comprising incubating the at least one modified magnetic buffer with the elution buffer for a time period sufficient to unbind the at least one nucleic acid from the at least one modified magnetic particle, and wherein the elution buffer is chosen from water and solutions comprising at least one buffer compound and/or at least one ion chelating agent.

11. A kit for nucleic acid purification, comprising:
(a) a suspension buffer comprising at least one first ion chelating agent, present in a concentration ranging from about 1 mM to about 10 mM, and at least one first buffer compound, present in a concentration ranging from about 10 mM to about 100 mM;
(b) a lysis buffer comprising at least one detergent chaotropic agent, present in a concentration ranging from about 1% to about 10% by weight, and at least one second buffer compound, present in a concentration greater than or equal to about 0.2 M;
(c) a binding buffer comprising at least one first chaotropic agent, present in a concentration ranging from about 4 M to about 6 M, optionally at least one first salt, present in a concentration ranging from about 0.1 M to about 2 M, at least one first alcohol, present in a concentration ranging from about 1% to about 5% by volume, and at least one third buffer compound, present in a concentration ranging from about 0.25% to about 3% by weight;
(d) a first wash buffer comprising at least one second chaotropic agent, present in a concentration ranging from about 4.5 M to about 6 M, at least one second ion chelating agent, present in an concentration ranging from about 1 mM to about 10 mM, at least one second alcohol, present in a concentration ranging from about 30% to about 50% by volume, and at least one fourth buffer compound, present in a concentration ranging from about 10 mM to about 100 mM;
(e) a second wash buffer comprising at least one third alcohol and optionally at least one second salt; and
(f) at least one magnetic particle.

12. The kit of claim 11, wherein the sample comprising the at least one nucleic acid is at least one bacterial cell comprising plasmid DNA.

13. The kit of claim 11, wherein the at least one first ion chelating agent is chosen from ethylenediamine tetraacetic acid (EDTA); ethylenediamine-N,N′-disuccinic acid (EDDS); cyclohexane-1,2-diaminetetraacetic acid (CDTA); iminodisuccinic acid (IDS); polyaspartic acid (PASA); methylglycinediacetic acid (MGDA); L-glutamic acid N,N-diacetic acid, tetrasodium salt (GLDA), and combinations thereof, and
wherein the at least one first buffer compound is chosen from Tris, Tris-HCl, 3-(N-morpholino)propanesulfonic acid (MOPS), 4-(2-hydroxyethyl)-1-piperazineethanesulfonic acid (HEPES), 2-(N-morpholino)ethanesulfonic acid (MES), and combinations thereof.

14. The kit of claim 11, wherein the at least one detergent chaotropic agent is chosen from sodium dodecyl sulfate (SDS), sodium dodecylbenzene sulfonate (SDBS), ammonium lauryl sulfate (ALS), potassium dodecyl sulfate (PDS), sodium myreth sulfate, octylphenol ethoxylates, polyoxyethylene sorbitan monolaureates, and combinations thereof, and
wherein the at least one second buffer compound is chosen from sodium hydroxide (NaOH), lithium hydroxide (LiOH), potassium hydroxide (KOH), rubidium hydroxide (RbOH), cesium hydroxide (CsOH), and combinations thereof.

15. The kit of claim 11, wherein the at least one first chaotropic agent is chosen from guanidine hydrochloride (GuHCl), guanidium thiocyanate (GuSCN), urea, and combinations thereof,
wherein the at least one first salt is chosen from ammonium sulfate ((NH₄)₂SO₄), ammonium acetate (NH₄Ac), lithium acetate (LiAc), potassium acetate (KAc), sodium acetate (NaAc), sodium chloride (NaCl), and combinations thereof,
wherein the at least one first alcohol is chosen from isopropanol, ethanol, methanol, butanol, and combinations thereof, and
wherein the at least one third buffer compound is chosen from glacial acetic acid, hydrochloric acid, and combinations thereof.

16. The kit of claim 11, wherein the at least one second chaotropic agent is chosen from guanidine hydrochloride (GuHCl), guanidium thiocyanate (GuSCN), urea, and combinations thereof,
wherein the at least one second ion chelating agent is chosen from ethylenediamine tetraacetic acid (EDTA); ethylenediamine-N,N′-disuccinic acid (EDDS); cyclohexane-1,2-diaminetetraacetic acid (CDTA); iminodisuccinic acid (IDS); polyaspartic acid (PASA); methylglycinediacetic acid (MGDA); L-glutamic acid N,N-diacetic acid, tetrasodium salt (GLDA), and combinations thereof,
wherein the at least one second alcohol is chosen from isopropanol, ethanol, methanol, butanol, and combinations thereof, and
wherein the at least one fourth buffer compound is chosen from Tris, Tris-HCl, 3-(N-morpholino)propanesulfonic acid (MOPS), 4-(2-hydroxyethyl)-1-piperazineethanesulfonic acid (HEPES), 2-(N-morpholino)ethanesulfonic acid (MES), and combinations thereof.

17. The kit of claim 11, wherein the at least one third alcohol is chosen from isopropanol, ethanol, methanol, butanol, and combinations thereof, and
wherein the at least one second salt is chosen from ammonium sulfate ((NH₄)₂SO₄), ammonium acetate (NH₄Ac), lithium acetate (LiAc), potassium acetate (KAc), sodium acetate (NaAc), sodium chloride (NaCl), and combinations thereof.

18. The kit of claim 11, wherein the at least one magnetic particle is chosen from carboxyl coated paramagnetic particles, silica-based paramagnetic particles, and combinations thereof.

19. The kit of claim 11, further comprising (g) an elution buffer chosen from water and solutions comprising at least one buffer compound and/or at least one ion chelating agent.

20. A kit for nucleic acid purification, comprising:
(a) a suspension buffer comprising at least one first ion chelating agent chosen from EDTA, isomers thereof, and combinations thereof, present in a concentration ranging from about 8 mM to about 10 mM, and at least one first buffer compound chosen from Tris, Tris-HCl and combinations thereof, present in a concentration ranging from about 10 mM to about 40 mM;
(b) a lysis buffer comprising at least one detergent chaotropic agent chosen from SDS, SDBS, and combinations thereof, present in a concentration ranging from about 1% to about 3% by weight, and at least one second buffer compound chosen from akali hydroxides and combinations thereof, present in a concentration greater than or equal to about 0.2 M;
(c) a binding buffer comprising at least one first chaotropic agent chosen from GuHCl, GuSCN, and combinations thereof, present in a concentration ranging from about 4 M to about 4.5 M, at least one first salt chosen from ammonium sulfate, ammonium acetate, sodium acetate, and combinations thereof, present in a concentration ranging from about 0.3 M to about 1.5 M, from about 1% to about 2% by volume of isopropanol, and at least one third buffer compound chosen from glacial acetic acid, hydrochloric acid, and combinations thereof, present in a concentration ranging from about 1% to about 3% by weight;
(d) a first wash buffer comprising at least one second chaotropic agent chosen from GuHCl, GuSCN, and combinations thereof, present in a concentration ranging from about 4.5 M to about 5 M, at least one second ion chelating agent chosen from EDTA, isomers thereof, and combinations thereof, present in an concentration ranging from about 1 mM to about 3 mM, from about 30% to about 40% by volume of isopropanol, and at least one fourth buffer compound chosen from Tris, Tris-HCl, and combinations thereof, present in a concentration ranging from about 20 mM to about 50 mM;
(e) a second wash buffer comprising at least about 70% by volume of ethanol and at least one second salt chosen from ammonium sulfate, ammonium acetate, sodium acetate, and combinations thereof, present in a concentration ranging from about 80 mM to about 100 mM; and
(f) at least one silica-based magnetic particle.
